(19) United States
(12) Patent Application Publication
(10) Pub. No.: US 2022/0301700 A1
(43) Pub. Date: Sep. 22, 2022

(54) METHOD FOR ALLOCATION OF AN APPOINTMENT

(71) Applicant: Siemens Healthcare GmbH, Erlangen (DE)

(72) Inventor: Martin HAMMES, Erlangen (DE)

(73) Assignee: Siemens Healthcare GmbH, Erlangen (DE)

(21) Appl. No.: 17/698,038

(22) Filed: Mar. 18, 2022

(30) Foreign Application Priority Data
Mar. 22, 2021 (DE) 10 2021 202 760.1

(51) Int. Cl. G16H 40/20; G16H 80/00; G16H 10/60; G06Q 10/10

(52) U.S. Cl. CPC G16H 40/20; G16H 80/00; G16H 10/60; G06Q 10/1095

(57) ABSTRACT

A computer-implemented method for allocation of an appointment comprises: receiving appointment information, wherein the appointment information includes information about a location of the appointment; receiving at least one first item of patient information of a first patient, wherein the first item of patient information includes information about a position of the first patient; determining a first distance between the location of the appointment and the position of the first patient; determining a first appointment invitation as a function of the first distance; and providing the first appointment invitation.

### CROSS-REFERENCE TO RELATED APPLICATION(S)

The present application hereby claims priority under 35 U.S.C. § 119 to German Patent Application No. DE 102021202760.1, filed Mar. 22, 2021, the entire contents of which are hereby incorporated herein by reference.

### BACKGROUND

Typically, a patient arranges an appointment with medical staff in advance of the appointment. The appointment can relate, for example, to an examination and/or to a medical intervention and/or treatment, etc. In particular, the appointment can relate to administering of a vaccine to the patient by the medical staff. It is known that appointments often have to be agreed a long time in advance and that there are long waiting times for an appointment. The long waiting times can be caused, in particular, by a lack of capacity of the medical staff and/or a medical-technical device used by the medical staff and/or of the vaccine.

It is known that the situation can occur where the appointment is cancelled by the patient on short notice. In other words, the situation can occur where the appointment is cancelled by the patient, for example, one day or a few hours before the appointment is due to take place. Unused capacities of the medical staff and/or of the medical-technical device used by the medical staff and/or of the vaccine occur in this way. This results, in particular, in unnecessary costs. In particular, a perishable product provided for the appointment, for example the vaccine, can become unstable if the appointment does not take place or if it is not administered at the appointment or is not administered promptly. This results in unnecessary material consumption, which is to be avoided in particular in the case of shortages of materials. It is thus of interest to allocate such appointments that have become free on short notice to another patient.

Overbooking of appointments is known in order to avoid unused capacities. This method is known in particular from the airline industry. With overbooking, an appointment can be allocated multiple times in order to minimize the risk that no patient turns up for the appointment. However, the situation can occur where more than one patient turns up for the appointment and thus at least one patient cannot be treated or examined. Alternatively or in addition, in a time interval of, for example, one day, more patients can be invited than there are appointments available. This can result in at least one patient having to wait for an unnecessarily long time for their appointment in the time interval. In particular, this can be the case if more patients actually turn up inside the time interval than there are appointments available. In particular in a waiting area of a doctor's practice and/or a hospital and/or a vaccination center, this can result in unnecessary exposure of the patient to infectious diseases. In particular, an infectious patient also constitutes a danger to other patients. For this reason the stay of the patient in the waiting area should be kept as short as possible.

Alternatively it is known to create a waiting list of patients. The medical staff can then contact at least one patient according to the waiting list if an appointment is cancelled or becomes free on short notice. This is time-consuming and staff-intensive, however, since the at least one patient has to be contacted manually. In addition, it can be necessary to contact a plurality of patients on the waiting list in order to find a patient who has time on short notice to use the appointment. In particular, such a waiting list is typically created independently of whether the patient is even in the vicinity at the time of the appointment. If the patient on the waiting list is on vacation for example, the medical staff have to contact the next patient on the waiting list. This is time-intensive and cost-intensive in particular.

### SUMMARY

One or more embodiments of the present invention provide a method for allocation of an appointment, which makes it possible to allocate an appointment automatically and promptly.

One or more embodiments provide a method for allocation of an appointment, by an appointment allocation unit for allocation of an appointment, by a computer program product and by a computer-readable storage medium. Advantageous developments are presented in the following description.

Below, embodiments are described both in respect of apparatuses and in respect of methods. Features, advantages or alternative embodiments mentioned in this connection can likewise also be transferred to the other claimed subject matter, and vice versa. In other words, the physical claims (which are directed, for example, toward an apparatus) can also be developed with the features which are described or claimed in conjunction with a method. The corresponding functional features of the method are formed by corresponding physical modules.

One or more embodiments relate to a computer-implemented method for allocation of an appointment. The method includes a method step of receiving appointment information. The appointment information includes information about a location of the appointment. The method also includes a method step of receiving at least one item of first item of patient information of a first patient. The first item of patient information includes information about a position of the first patient. The method also includes a method step of determining a first distance between the location of the appointment and the position of the patient. The method also includes a method step of determining a first appointment invitation as a function of the first distance. The method also includes a method step of providing the first appointment invitation.

The appointment can be, in particular, an appointment with a doctor and/or in a hospital and/or at a vaccination center. In other words, the appointment can be a medical appointment. In particular, the appointment can relate to an appointment of a patient with medical staff. The medical staff can be, for example, a doctor or a radiologist or a medical assistant. For example, during the appointment an examination and/or a diagnosis can be carried out or made. Alternatively, a medical image of the patient can be created during the appointment. The medical image can be, for example, a magnetic resonance scan or a computed tomography image. Alternatively, a vaccine can be administered to the patient during the appointment. In other words, the appointment can be a vaccination appointment.

Alternatively, the appointment can be any other appointment. For example, the appointment can be an appointment of a customer or client with a service provider. For example, the appointment can be an appointment in a citizen center. Alternatively, the appointment can be an appointment for shopping. Alternatively, the appointment can be an appointment of a client with an attorney. Alternatively, the appointment can be an appointment in a car repair shop.

The appointment is, in particular, a free appointment. In other words, the appointment has not yet been assigned to a patient. In particular, the appointment may have become free on short notice. In other words, a different patient may have cancelled the appointment on short notice. On short notice can mean, for example, that the appointment was cancelled one day or a few hours before it should have taken place.

In the method step of receiving the appointment information, the appointment information is received via an interface. The appointment information relates to the appointment to be allocated. In other words, the appointment information includes information relating to the appointment which is to be allocated. The appointment information includes information about the location of the appointment. In other words, the appointment information includes information about where the appointment takes place. The appointment information can include the information about the location of the appointment in the form of an address. Alternatively, the appointment information can include the information about the location of the appointment in the form of Global Positioning System coordinates (acronym: GPS coordinates). In particular, the appointment information can include the GPS coordinates in a GPS Exchange Format (acronym: GPX). In other words, the appointment information can include the GPS coordinates in a GPX file.

In the method step of receiving the first item of patient information of the first patient, the first item of patient information is received by the interface. The first item of patient information includes information about the first patient. The first patient can be, in particular, a human or a person who is waiting for an appointment. The first patient can be, in particular, a person who could potentially use the appointment. In other words, the first patient can be a person who can possibly use the appointment on short notice. In particular, the first patient can be registered to be informed about an appointment that has become free on short notice. In particular, the first patient can indicate by way of the registration that they would like to be considered for the allocation of an appointment. The first item of patient information includes information about the position of the first patient. The position of the first patient can be, in particular, the domicile or the place of work or the main place of residence of the first patient. Alternatively or in addition, the position of the first patient can describe a current position of the first patient. In other words, the position of the first patient can indicate where the first patient is residing at the time of receiving the first item of patient information. In particular, the position of the first patient can indicate where the first patient is residing at the time at which the appointment is to be allocated. The first item of patient information can include the information about the position of the patient in the form of an address and/or in the form of GPS coordinates. In particular, the first item of patient information can include the position of the patient the GPS coordinates in the form of a GPX file. The information about the position of the first patient can be provided, in particular, by a mobile device belonging to the first patient. The mobile device can be, in particular, a cell phone or smartphone or mobile phone and/or a tablet. In particular, the position of the first patient can be determined via location determination of the mobile device. Alternatively or in addition, a radio cell, which the mobile device of the first patient is dialed into, can be assumed as the position of the patient. In particular, the first item of patient information can include the information about the position of the first patient in the form of GPS coordinates and/or in the form of data about a dial-in into a mobile network.

In the method step of determining the first distance, the first distance between the location of the appointment and the position of the first patient is determined. In particular, the first distance can indicate a spacing or a route length between the location of the appointment and the position of the first patient. In other words, the first distance describes how far the first patient is from the location of the appointment. The first distance can be indicated, in particular, in kilometers. The first distance can be determined, in particular as linear distance between the location of the appointment and the position of the patient. Alternatively, the first distance can indicate an actually drivable or walkable route length between the position of the patient and the location of the appointment. In other words, the first distance can indicate how many kilometers the first patient has to cover from their position in order to arrive at the location of the appointment. If the first distance describes the linear distance between the position of the first patient and the location of the appointment, the first distance can be determined, in particular, on the basis of the GPS coordinates and/or the address of the position of the first patient and of the location of the appointment. If the first distance indicates the actually drivable or walkable route length, the first distance can be determined on the basis of the GPS coordinates or the address and using a geoinformation system. For example, the first distance can be determined on the basis of Google Maps and/or using Open Street Maps. Actually drivable or walkable roads or paths between the position of the first patient and the location of the appointment can be considered when determining the first distance.

In the method step of determining the first appointment invitation, the first appointment invitation is determined as a function of the first distance. The first appointment invitation can be an invitation for the first patient to use the appointment. The first appointment invitation can include, in particular, the appointment information. In particular, the first appointment invitation can include the information about the location of the appointment and/or information about a start time of the appointment. Alternatively or in addition, the appointment invitation can include information about a duration of the appointment.

The first appointment invitation is determined as a function of the first distance. In particular, the first appointment invitation is only determined when the first distance is not too great. In other words, the first appointment invitation is only determined if the first patient can be at the appointment at the location of the appointment promptly. In other words, if the first appointment invitation is only determined when the first patient can reach the location of the appointment in a particular time. In particular, the first appointment invitation is determined when the first distance undershoots a first threshold value.

In the method step of providing the first appointment invitation, the first appointment invitation is provided via the interface. In particular, the first patient is provided with the first appointment invitation. In particular, the first patient can be provided with the first appointment invitation via their mobile device. In particular, the first item of appointment information can be provided in the form of a pop-up window. Alternatively, the first patient can be provided with the first appointment invitation by SMS and/or e-mail.

The inventors have found that it is advantageous to determine the first distance of the first patient from the location of the appointment in advance. This can ensure that only the patient who can reach the location of the appointment within a particular time receives a first appointment invitation. The inventors have also found that it is thus possible to prevent an unnecessary number of patients from being contacted who cannot arrive, or cannot arrive promptly, at the location of the appointment.

According to one aspect of embodiments of the present invention, the first item of patient information includes information about the mobility of the first patient. The method steps of determining the first appointment invitation and of providing the first appointment invitation only take place when the first distance undershoots a first threshold value. The first threshold value depends on the mobility of the first patient in this case.

The mobility of the first patient can describe with which manner of transport the first patient can arrive at the appointment. In other words, the mobility of the first patient describes which manner of transport the first patient has available to them to arrive at the location of the appointment. The manner of transport can be, for example, a bicycle, a bus, a train, a car, a taxi, etc. Alternatively or in addition, the information about the mobility of the first patient can indicate that the patient can come on foot to the location of the appointment. The information about the mobility of the first patient can alternatively or in addition include information about how quickly the first patient can arrive at the location of the appointment.

Alternatively or in addition, the mobility can describe an individual mobility of the first patient. For example, the individual mobility can indicate whether the first patient is a wheelchair user, whether they have difficulty walking, etc. In other words, the individual mobility can indicate whether the first patient is limited in terms of their mobility compared to a mean of the population.

The information about the mobility can be provided by the first patient. Alternatively or in addition, the information can be derived from health data or patient data of the first patient. For example, the information about the mobility can be derived from the age of the first patient. Alternatively or in addition, the information about the mobility of the first patient can be derived from a pre-existing condition of the first patient, etc. In particular, the information about the mobility of the first patient can be empirically ascertained.

The first threshold value then depends on the mobility of the first patient. In particular, the first threshold value can thus be patient-specific. The first threshold value is in particular defined in such a way that the first patient can arrive at the location of the appointment within a particular journey time interval. In other words, the first threshold value indicates a stretch or distance, which the first patient can cover in the journey time interval depending on their mobility. The first patient can cover a greater distance with a car than on foot within the journey time interval. A timetable of the bus and/or the train can also be considered with the first threshold value. In other words, the first threshold value is defined or determined or chosen in such a way that it is possible to ensure that the first patient can be at the location of the appointment within the journey time interval. The journey time interval can depend on a current time and a time at which the appointment should take place. The current time corresponds to the time at which the first appointment invitation is provided.

The first appointment invitation is only determined and provided when the first distance undershoots the first threshold value. In other words, the first appointment invitation is only determined and provided when it has been possible to ensure that the first patient can be at the location of the appointment within the journey time interval or up to a particular time.

The inventors have found that it is thereby possible to ensure that only those patients, who can be at the location of the appointment promptly, receive an appointment invitation. Patients, who, for example, are currently on vacation and thus more remote from the location of the appointment, do not receive an appointment invitation. The inventors have also found that more mobile patients can also be invited when they are currently further away than less mobile patients. The inventors have found that, as a function of the current position of the patient, in this way only those patients who can possibly use the appointment receive an appointment invitation. The inventors have also found that in this way it is possible to save time and costs. The inventors have also found that in this way it is possible to cater to the individual mobility of the first patient. In other words, a patient, who is limited in terms of their mobility, can be provided with more time to arrive at the appointment than another patient not limited in terms of their mobility.

According to a further aspect of embodiments of the present invention, the method includes a method step of receiving confirmation information if the first patient accepts the first appointment invitation.

The confirmation information can be provided by the first patient. The confirmation information can be provided, in particular, by the first patient in response to the first appointment invitation. The confirmation information can be provided, for example, via an input by the first patient on their mobile device. The first patient can confirm the first appointment invitation in particular via a user interface (UI) or by e-mail or SMS or telephone.

With the confirmation information, information is received to the effect that the first patient has accepted the first appointment invitation. In particular, the confirmation information thus provides information that it was possible to successfully allocate the appointment.

The confirmation information can include information about the first patient. For example, the confirmation information can include information about the age or the gender of the first patient. In particular, the confirmation information can include information about at least one risk factor of the first patient, for example about a pre-existing condition. In particular, with the confirmation information the medical staff can be informed about whether particular precautions have to be taken for the appointment.

The inventors have found that through the confirmation information, information can be received to the effect that the appointment has been allocated. In addition, the inventors have also found that the medical staff is informed by way of the confirmation information about the appointment having been allocated. The medical staff can thus prepare themselves for the appointment.

According to a further aspect of embodiments of the present invention, the appointment relates to dispensing of a vaccine.

In other words, the vaccine can be administered at the appointment to a patient, for example the first patient. In other words, a patient, for example the first patient, can be vaccinated with the vaccine at the appointment. The vaccine can be designed, in particular, to protect the vaccinated patient against an infectious disease. In particular, the probability of an infection of the vaccinated patient with the infectious disease can be reduced via the vaccine. In particular, a probability of a severe progression of the infectious disease can be reduced. For example, the vaccine can be a vaccine against SARS-CoV-2. In other words, the infectious disease can be Covid-19, which is triggered by Corona virus SARS-CoV-2.

The location of the appointment can be, for example, a vaccination center. Alternatively or in addition, the location of the appointment can be a family practice and/or a hospital and/or a pharmacy and/or a workplace and/or a care facility, etc. The appointment can include administering of the vaccine and an observation time of the patient.

The inventors have found that the method is suitable for coordinating or optimizing vaccine distribution.

According to a further aspect of embodiments of the present invention, the appointment information includes information about the type of appointment. The first item of patient information includes information about the type of appointment sought. The method also includes a method step of comparing the type of appointment and the type of appointment sought. The method steps of determining the first distance, of determining the first appointment invitation and of providing the first appointment invitation are carried out if the comparison produces a match.

The type of appointment indicates, in particular, what will be done at the appointment. For example, the type of appointment can indicate “Vaccination with the vaccine” or “First vaccination with the vaccine” or “Second vaccination with the vaccine” or “Dentist appointment check-up” or “Preventive check-up” or “Thorax image magnetic resonance scan”, etc. Alternatively or in addition, the type of appointment can indicate with whom or where the appointment will take place. For example, the type of appointment can indicate “Vaccination center” or “Dentist” or “Internist” or “Cardiologist”, etc.

The type of appointment sought can be formed analogously.

The type of appointment can be chosen, in particular, by the physician or the medical assistant from a list and be added to the appointment information. The type of appointment sought can be chosen, in particular, by the first patient from a list and be added to the first item of patient information. The type of appointment or the type of appointment sought can be added in the form of a code to the appointment information or the first item of patient information. In other words, a unique code can be assigned to any possible type of appointment. The code for the type of appointment or for the type of appointment sought is then incorporated by the appointment information or the first item of patient information. The code can be indicated, for example, in the form of the International Statistical Classification of Diseases and Related Health Problems (acronym: ICD) or be based on it. Alternatively or in addition, the type of appointment or the type of appointment sought can be added in the form of a free text by the physician or the medical assistant or the first patient to the appointment information or the first item of patient information.

In the method step of comparison of the type of appointment and the type of appointment sought, the type of appointment is compared with the type of appointment sought. Comparing can be based on the terms chosen from the list. Alternatively, comparing can be based on the code.

Alternatively, comparing can be based on Natural Language Processing (NLP) if the type of appointment or the type of appointment sought is incorporated by the appointment information or by the first item of patient information in the form of a free text.

The method steps of determining the first distance, of determining the first appointment invitation and of providing the first appointment invitation are only carried out if the type of appointment matches the type of appointment sought.

The inventors have found that in this way it is possible to ensure that the first patient is also only offered an appointment which they are actually seeking. The inventors have also found that in this way it is possible to manage or coordinate different types of appointments. The inventors have also found that in this way it is possible to save time since only patients who are actually interested are invited. In particular, a patient can register for a plurality of different appointments in the same way. The manageability for the patient is improved as a result.

According to a further aspect of embodiments of the present invention, the first appointment invitation is provided in the method step of providing the first appointment invitation for the duration of a predefined period.

In other words, the first appointment invitation is valid only for the duration of the predefined period. In particular, the confirmation information can only be received within this period. In particular, the first patient can only accept the appointment invitation within the period. In particular, the first patient can only trigger receiving of the confirmation information within the predefined period by accepting the first appointment invitation.

The predefined period can be selected in such a way that from the instant at which the predefined period expires, the first patient also has adequate time to arrive at the location of the appointment. Alternatively or in addition, the predefined period can be chosen in such a way that the physician and/or the medical assistant has adequate time between the instant of expiry of the predefined period and the appointment to prepare for the appointment. Alternatively or in addition, the predefined period can be chosen in such a way that once the predefined period has expired, adequate time remains to provide a different patient with an appointment invitation if the first patient does not accept the first appointment invitation.

If the first appointment invitation was provided via a pop-up window, the pop-up window can disappear after the predefined period has expired. In particular, the remaining time of the predefined period can be rundown in the pop-up window. Alternatively or in addition, the first patient can be informed via an SMS or e-mail that the predefined period has expired. Alternatively or in addition, confirmation information is no longer accepted or received once the predefined period has expired.

The inventors have found that in this way it is possible to ensure that the preparation time and/or the journey time to the appointment is adequate for the appointment to be able to take place as planned. In addition, the inventors have found that in this way it is possible to prevent an appointment that has already elapsed or an appointment in the past from being subsequently accepted or accepted after expiry of the appointment. Ambiguities can thus be avoided.

According to a further aspect of embodiments of the present invention, the appointment information includes information about a time at which the appointment takes place. The first appointment invitation includes the information about the location of the appointment and/or about the time at which the appointment takes place.

The information about the time at which the appointment takes place can include, in particular, an instant at which the appointment begins. Alternatively, the information about the time at which the appointment takes place can include a period within which the appointment takes place. In particular, the period can indicate a duration of the appointment.

The first appointment invitation can then be determined in the method step of determining the first appointment invitation as a function of the appointment information. The first appointment invitation can be determined in particular in such a way that it includes the information about the location of the appointment. Alternatively or in addition, the first appointment invitation can be determined in such a way that it includes the information about the time at which the appointment takes place.

The inventors have found that the first patient requires information about the location of the appointment and/or about the time at which the appointment takes place in order to decide whether they can accept the appointment. In addition, the inventors have also found that the first patient requires information about the location of the appointment and/or about the time at which the appointment takes place in order to use the appointment.

According to a further aspect of embodiments of the present invention, the method also includes a method step of receiving at least one second item of patient information of a second patient. The second item of patient information includes information about a position of the second patient. The method also includes a method step of determining a second distance between the location of the appointment and the position of the second patient.

The second patient is in particular different from the first patient. The second patient can in particular likewise have an interest in the appointment. The second item of patient information can be formed in accordance with the description relating to the first item of patient information. The second distance can be determined analogously to the first distance.

The inventors have found that with the method, more than one patient can be informed about the appointment. The inventors have also found that in this way it is possible to increase the probability that a patient will accept the appointment or the invitation to the appointment.

According to a further optional aspect of embodiments of the present invention, the second item of patient information includes information about a type of appointment sought. In the method step of comparison of the type of appointment and the type of appointment sought, the type of appointment according to the appointment information is also compared with the type of appointment sought according to the second item of patient information.

The type of appointment sought according to the first item of patient information can be different from the type of appointment sought according to the second item of patient information. Alternatively, the type of appointment according to the first item of patient information can be identical to the type of appointment sought according to the second item of patient information. In particular, the type of appointment sought of the second item of patient information can indicate which type of appointment the second patient is seeking or for which type of appointment the second patient is waiting. In particular, the method step of determining the second distance can only be carried out when the method step of determining produces a match in the type of appointment and the type of appointment sought according to the second item of patient information.

The inventors have found that in this way a plurality of different appointments can be coordinated for a plurality of patients. In particular, in this way it is possible to ensure that an invitation for an appointment which matches an appointment sought by a patient is provided.

According to a further aspect of embodiments of the present invention, the method also includes a method step of determining a second appointment invitation as a function of the second distance. The method also includes a method step of providing the second appointment invitation.

The method step of determining the second appointment invitation can be formed analogously to the method step of determining the first appointment invitation.

The method step of providing the second appointment invitation can be formed analogously to the method step of providing the first appointment invitation. In particular, in the method step of providing the second appointment invitation, the second patient is provided with the second appointment invitation. In particular, the second patient can be provided with the second appointment invitation for example via a pop-up window or an SMS or an e-mail. In particular, the second patient can be provided with the second appointment invitation in a different way to how the first patient is provided with the first appointment invitation. In particular, providing the appointment invitations can be patient-specific.

In particular, the method steps of determining the second appointment invitation and of providing the second appointment invitation can be carried out as a function of the comparison of the type of appointment and the type of appointment sought according to the second item of patient information. In particular, the method steps of determining the second appointment invitation and of providing the second appointment invitation can only be carried out when the comparison produces a match.

The inventors have found that more than one patient can be provided with an invitation to the same appointment. In particular, more than one patient can be provided with an invitation to the same appointment if more than one patient is eligible to use the appointment. The eligibility depends on the distance between the position of the patient and the location of the appointment and/or on the type of appointment.

According to a further optional aspect of embodiments of the present invention, the second appointment invitation is provided in the method step of providing the second appointment invitation for the duration of a predefined period.

The duration of the predefined period can be formed according to the above description. In particular, the predefined period can be patient-specific. In particular, the predefined period can depend on the mobility of the patient. In particular, the predefined period in which the first appointment invitation is provided can thus be different from the predefined period in which the second appointment invitation is provided. Alternatively, the predefined period can be the same length for all appointment invitations.

According to a further optional aspect, the second appointment invitation includes information about the location of the appointment and/or about the time at which the appointment takes place.

The information about the location of the appointment and the time at which the appointment takes place can be formed in particular as described above.

In particular, the information about the location of the appointment can indicate where the appointment takes place. For example, the information about the location of the appointment can indicate the location of the appointment in the form of an address and/or in the form of GPS coordinates. In particular, the second appointment invitation can include a GPX file with the GPS coordinates of the location of the appointment as information about the location of the appointment.

The information about the time at which the appointment takes place can indicate, in particular, when the appointment takes place. In particular, the information about the time at which the appointment takes place can indicate when the appointment begins. Alternatively or in addition, the information about the time at which the appointment takes place can indicate how long the appointment lasts. In other words, the information about the time at which the appointment takes place can include information about the duration of the appointment.

According to a further aspect of embodiments of the present invention, the method steps of determining the second appointment invitation and of providing the second appointment invitation only take place when the second distance undershoots a second threshold value. The second threshold value depends on the mobility of the second patient.

The mobility of the second patient is formed as described above in respect of the first patient. The second threshold value is thus formed in such a way that the second patient can cover the distance defined by the second threshold value within a particular journey time interval, as a function of the manner of transport that are available to them. The journey time interval can be chosen, in particular, in such a way that the second patient can be at the location of the appointment at the time at which the appointment takes place. The second appointment invitation is thus only determined and provided when it has been possible to ensure that the second patient can turn up promptly at the appointment.

The second threshold value can, in particular, be different from the first threshold value. In particular, the first and the second threshold values can be patient-specific. Alternatively, the first and the second threshold value can be identical.

The inventors have found that, advantageously, only those patients are invited who also have the possibility of turning up promptly to the appointment. The inventors have also found that this depends in particular on the mobility of the patient. The inventors have also found that a patient who can arrive at the appointment by car in the time up to the beginning of the appointment can cover a greater distance than a patient who comes on foot. In particular, the threshold value can thus be chosen or determined accordingly. The inventors have found that the threshold value can be patient-specific.

According to a further aspect of embodiments of the present invention, the confirmation information is received in the method step of receiving the confirmation information if the second patient accepts the appointment invitation.

In other words, the confirmation information is also received when the second patient accepts the appointment. In particular, the confirmation information is received if at least one, the first and/or the second patient, accepts the first or second appointment invitation. The confirmation information indicates whether the first and/or the second patient has accepted the first or second appointment invitation. In particular, the confirmation information can include information about which patient accepted the first or second appointment invitation. The confirmation information can be provided, for example, via an input of the second patient on their mobile device. The second patient can confirm the second appointment invitation in particular via a user interface (UI) or by e-mail or SMS or telephone.

The inventors have found that on the basis of the confirmation information it is possible to see whether the appointment could be allocated. The inventors have also found that from receiving of the confirmation information it is possible to derive that no further patients should be contacted in order to allocate the appointment. In other words, it is possible to derive from the confirmation information that the appointment is no longer available. In addition, it is possible to derive from the confirmation information whether the physician and/or the medical assistant should prepare for the appointment or not.

According to an optional aspect of embodiments of the present invention, all provided appointment invitations can be withdrawn as soon as the confirmation information is received from a patient.

In other words, the patients who have received an appointment invitation are informed that the appointment has been allocated if the confirmation information has been received. Withdrawal can in this case mean the pop-up window disappears. Alternatively, the first and/or second appointment invitation can be withdrawn by sending a further SMS and/or e-mail to the first or second patient. The SMS and/or e-mail includes information to the effect that the appointment is no longer available.

The inventors have found that the confirmation information makes it possible to prevent an appointment from being allocated multiple times.

According to a further aspect of embodiments of the present invention, the method includes a method step of providing allocation information as soon as the confirmation information has been received.

The first and/or second patient can be provided with the allocation information in particular via an SMS and/or an e-mail and/or a pop-up window. In particular, providing the allocation information can include a withdrawal of the first and/or second appointment invitation. In particular, providing the allocation information can include the pop-up window, by which the first and/or second item of appointment information is provided, disappearing. In particular, the first and/or second patient can be informed with the allocation information that the appointment has already been allocated. After providing the allocation information it is, in particular, no longer possible to accept or confirm the appointment or the first or second appointment invitation.

In particular the patient who has not accepted the corresponding appointment invitation can be provided with the allocation information. For example, the second patient can be provided with the allocation information if the first patient accepts the first appointment invitation and the confirmation information has been received. Alternatively, the first patient can be provided with the allocation information if the second patient accepts the second appointment invitation and the confirmation information has been received.

If the first and the second patient accept the first or second appointment invitation simultaneously, a patient can be determined who should use the appointment. The other patient can then be provided with the allocation information.

If more than one patient accepts the corresponding appointment invitation simultaneously, a patient can be determined who may use the appointment. All other patients are provided with the allocation information. The patient who may use the appointment can be determined on the basis of a random principle. Alternatively, the patient who may use the appointment can be determined on the basis of a prioritization. The prioritization can be based, for example, on how long the patient has already been waiting for the appointment. Alternatively or in addition, the prioritization can be based on a medical prioritization. The medical prioritization can indicate, for example, patient groups, which divide the patients according to how necessary the appointment is for the corresponding patient. If the appointment relates, for example, to a vaccination, the medical prioritization can indicate that patients over 80 and/or patients with a pre-existing condition belong to a patient group with higher priority than healthy, young patients. The first and/or the second patient can be assigned to one of the patient groups. The assignment of the patients to the patient groups corresponds to the prioritization. In this case, with simultaneous acceptance by a plurality of patients, the patient who should preferably use the appointment according to the prioritization can be determined. In other words, the patient who is assigned to the patient group with the highest priority can be determined. All other patients can be provided with the allocation information.

In embodiments, the patient who has accepted the corresponding appointment invitation and who should use the appointment can also be provided with the allocation information. The allocation information can inform the patient that they have been successfully recorded or registered for the appointment. In other words, the allocation information can inform any patient about whether they should use the appointment or not and/or whether the appointment is still available or not. The allocation information can thus be configured to be patient-specific.

The inventors have found that through the allocation information, the patients can be informed if the appointment is no longer available or if, based on the prioritization, they should not use the appointment or it has not been determined that they may use the appointment.

According to a further aspect of embodiments of the present invention, the first item of patient information includes first item of prioritization information of the first patient in respect of the appointment. The second item of patient information includes a second item of prioritization information of the second patient in respect of the appointment. The method steps of determining the first appointment invitation, of providing the first appointment invitation, of determining the second appointment invitation and of providing the second appointment invitation take place as a function of the first and/or second item of prioritization information.

The prioritization information indicates, in particular, a priority of the corresponding patient in respect of the appointment. In other words, the prioritization information indicates how urgently the corresponding patient should receive an appointment for the corresponding treatment or examination or vaccine allocation. Groups of patients can be predefined for this. The prioritization information can then indicate a patient group to which the corresponding patient belongs or to which the corresponding patient is assigned. The patient groups can divide or distribute the patients for example according to their age and/or according to their pre-existing condition and/or according to their occupation and/or according to their waiting time for the appointment, etc. The division can be legally defined in particular. The patient groups can differ with regard to their prioritization in the appointment allocation. Membership of a patient group describes the priority of a patient. The first patient group should, for example, particularly preferably be offered an appointment, or be provided with an appointment invitation. The priority is then lower from patient group to patient group.

In particular, it is possible to specify which patient group or which patient groups is provided with an appointment invitation. In other words, only patients who belong to one of the specified patient groups or are incorporated by one of the specified patient groups can be provided with an appointment invitation. In particular, only a patient who belongs to the specified patient group or the specified patient groups is entitled to receive an appointment invitation. On the basis of the prioritization information it is possible to determine whether the corresponding patient belongs to one of the patient groups.

In particular, the first or second patient is provided with the first or second appointment invitation only when they are entitled to receive an appointment invitation according to the first item of prioritization information.

In particular, on the basis of the first or second item of prioritization information it is possible to determine which patient should preferably use the appointment if the first and the second patient simultaneously accept the first or second appointment invitation. In particular, the other patient can then be provided with the allocation information.

The inventors have found that, advantageously, only those patients who are entitled to use the appointment are informed about the appointment. In particular, the inventors have found that on the basis of the prioritization information it is possible to ensure that the patients who need the appointment more urgently than other patients can be preferentially treated.

According to a further aspect of embodiments of the present invention, the first and/or second item of prioritization information includes a prioritization of the first or second patient in respect of their age, their occupation, their pre-existing conditions and/or their social environment.

In other words, a priority, with which the first or second patient should use the appointment, depends on their age, their occupation, their pre-existing conditions and/or their social environment. In other words, the above-described division into patient groups is based on the age, the occupation, the pre-existing conditions and/or the social environment. In particular, the prioritization of the first or second patient thus depends on which patient group they belong to or which patient group incorporates them.

The first or second item of prioritization information can include the age, the occupation, the pre-existing conditions and/or the social environment of the first or second patient. Alternatively, the prioritization information can include information about to which patient group the corresponding patient was assigned on the basis of their age, their occupation, their pre-existing conditions and/or their social environment. In particular, the prioritization of the first or second patient can be derived from the first or second item of prioritization information.

In particular, the first or second item of prioritization information can include the age in the form of a date of birth of the first or second patient. Alternatively or in addition, the first or second item of prioritization information can include the age of the first or second patient in the form of a number, which indicates the number of years of life of the first patient or patients.

Alternatively or in addition, the first or second item of prioritization information can include, in particular, the occupation of the first or second patient in the form of a standardized occupational group. A standardized occupational group can be, for example, “Care”, “Teaching”, “Police force”, “Medical staff”, etc. Alternatively, the prioritization information can include a job title of the corresponding patient. In particular, the first or second patient of a patient group can then be assigned to the job title by Natural Language Processing.

The first or second item of prioritization information can include the pre-existing conditions of the first or second patient, for example in the form of ICD-10 (International Statistical Classification of Diseases and Related Health Problems).

Alternatively or in addition, the first or second item of prioritization information can include information about the social environment of the first or second patient. The information about the social environment of the patient can include, for example, information about whether the first or second patient has contact with a person from a more highly prioritized patient group. Alternatively or in addition, the information about the social environment can include information about whether the first or second patient has a large number of job-related contacts. Alternatively or in addition, the information about the social environment can comprise information about the housing situation of the first or second patient. For example, the housing situation can indicate how many people the first or second patient lives with in a household and under what conditions.

Alternatively, the first or second item of prioritization information can include information about which patient group the first or second patient belongs to according to their age, occupation, pre-existing conditions and/or social environment. In other words, the first or second item of prioritization information can include information about the prioritization of the first or second patient, which can be derived from the age, the occupation, the pre-existing conditions and/or the social environment.

The inventors have found that the prioritization of the first or second patient depends on different patient-specific conditions. The inventors have also found that these conditions can be considered on the basis of the prioritization information when providing the appointment invitations. The inventors have also found that the conditions can also be considered when determining the patient who should use the appointment if more than one patient simultaneously accepts the appointment invitation. The inventors have also found that the prioritization of the first or second patient can be derived from the first or second item of prioritization information. The inventors have also found that the first or second item of prioritization information can directly include the prioritization of the first or second patient, for example in the form of the associated patient group.

According to a further aspect of embodiments of the present invention, the first and/or second item of prioritization information includes information about an occurrence of infection of an infectious disease, which can be reduced with the vaccine, in an environment of the first or second patient.

In particular, the first and/or second item of prioritization information includes the information about the occurrence of infection if the appointment relates to dispensing of the vaccine. The vaccine can be formed as described above. The infectious disease can be formed as described above. The wording “the vaccine reduces the infectious disease” means that the probability of infection with the infectious disease and/or the probability of a severe progression of the infectious disease is reduced by the vaccine.

The occurrence of infection describes a spread of the infectious disease in the environment of the first or second patient. The information about the occurrence of infection can include an incidence and/or a number of daily new infections and/or a reproduction rate and/or intensive care bed occupation and/or a death rate.

The environment of the first or second patient can include, in particular, a typical radius of movement of the first or second patient. For example, the area can be defined by a commute and/or by a route to leisure activities, etc. In particular, the area then describes a region in which the first or second patient typically resides. In particular, the area can be patient-specific. Alternatively, the area can be rigidly defined. For example, the area can include an region with a radius of 10 or 15 or 20 or 50 km around the domicile of the first or second patient. Alternatively the area can include a town or a district in which the first or second patient lives.

The information about the occurrence of infection describes the occurrence of infection in the area of the first or second patient. The prioritization of the first or second patient can depend on the occurrence of infection. If the occurrence of infection is high and/or uncontrollable in the area of the first or second patient, the first or second patient can be given higher priority than if the occurrence of infection is low and/or controllable. A high occurrence of infection can be defined, for example, by a comparatively high number of new infections and/or by a high occupancy rate of intensive care beds and/or by a comparatively high reproduction rate, etc.

The inventors have found that dynamic factors such as the occurrence of infection can also be considered when allocating the appointment. The inventors have also found that in this way it is possible to ensure that the occurrence of infection can be optimally contained nationwide.

One or more embodiments of the present invention also relate to an appointment allocation unit for allocation of an appointment. The appointment allocation unit includes an interface and a computing unit. The interface is designed for receiving appointment information. The appointment information includes information about a location of the appointment. The interface is also designed for receiving at least a first item of patient information. The first item of patient information includes information about a position of the first patient. The computing unit is designed for determining a first distance between the location of the appointment and the position of the first patient. The computing unit is also designed for determining a first appointment invitation as a function of the first item of patient information and/or as a function of the first distance. The interface is also designed for providing the first appointment invitation as a function of the first item of patient information. The first item of appointment information is only issued if the first distance undershoots a first threshold value.

An appointment allocation unit of this kind can be designed, in particular, to carry out the above-described method for allocation of an appointment and its aspects. The appointment allocation unit is designed to carry out this method and its aspects in that the interface and the computing unit are designed to carry out the corresponding method steps.

One or more embodiments of the present invention also relate to an appointment allocation system for allocation of an appointment. The appointment allocation system includes an above-described appointment allocation unit. The appointment allocation system also includes at least one first patient terminal and one provision unit. The provision unit is designed for sending appointment information. The first patient terminal is designed for sending a first item of patient information of a first patient and for receiving a first appointment invitation.

The first patient terminal can be, in particular, a mobile device. In particular, the first patient terminal can be a smartphone and/or a tablet. Alternatively, the first patient terminal can be a mobile phone or cell phone and/or a computer or laptop. The first patient terminal can be designed, in particular, to display the first item of appointment information. The first appointment invitation can be displayed via a display unit of the patient terminal. The display unit can be, for example, a screen or a monitor. In embodiments of the present invention, the first patient terminal can be designed to receive a user input by the first patient. In particular, the first patient terminal can receive the user input via a user interface (acronym UI). For example, the user input can be received via a receiving unit of the first patient terminal. The receiving unit can be, for example, a computer mouse and/or a computer keyboard and/or a touchpad and/or a touchscreen.

The provision unit can be, in particular, a mobile device. For example, the provision unit can be a smartphone and/or a tablet. Alternatively, the provision unit can be a computer or a laptop. In particular, the provision unit can be designed to receive a user input by the medical staff. In particular, the first item of appointment information can then be provided or input by the medical staff. The user input can be received by the provision unit analogously to the description relating to the user input for the patient terminal. In particular, the provision unit can be designed to receive data from a server or send data to the server. The server can include a database for management of the appointments and/or the first and/or second item(s) of patient information.

One or more embodiments of the present invention also relate to a computer program product with a computer program and a computer-readable medium. An implementation largely in terms of software has the advantage that even previously used appointment allocation units can easily be retrofitted by way of a software update in order to work in the described manner. Apart from the computer program, a computer program product can optionally comprise additional constituent parts, such as documentation and/or additional components, as well as hardware components, such as hardware keys (dongles, etc.) in order to use the software.

In particular, embodiments of the present invention also relate to a computer program product with a computer program, which can be loaded directly into a memory unit of an appointment allocation unit, with program segments in order to carry out all steps of the above-described method for allocation of an appointment and its aspects when the program segments are executed by the appointment allocation unit.

In particular, embodiments of the present invention relate to a computer-readable storage medium on which program segments, which can be read and executed by an appointment allocation unit, are stored in order to carry out all steps of the above-described method for allocation of an appointment and its aspects when the program segments are executed by the appointment allocation unit.

### BRIEF DESCRIPTION OF THE DRAWINGS

The above-described properties, features and advantages of the present invention will become clearer and more comprehensible in conjunction with the following Figures and their descriptions. The Figures and descriptions are not intended to limit the present invention and its embodiments in any way.

Identical components are provided with corresponding reference numerals in different Figures. As a rule, the Figures are not to scale. In the drawings:

FIG. 1 shows a first example embodiment of a method for allocation of an appointment,

FIG. 2 shows a second example embodiment of a method for allocation of an appointment,

FIG. 3 shows a third example embodiment of a method for allocation of an appointment,

FIG. 4 shows a fourth example embodiment of a method for allocation of an appointment,

FIG. 5 shows a fifth example embodiment of a method for allocation of an appointment,

FIG. 6 shows a sixth example embodiment of a method for allocation of an appointment,

FIG. 7 shows an appointment allocation unit for allocation of an appointment, according to an example embodiment,

FIG. 8 shows an appointment allocation system for allocation of an appointment, according to an example embodiment.

### DETAILED DESCRIPTION

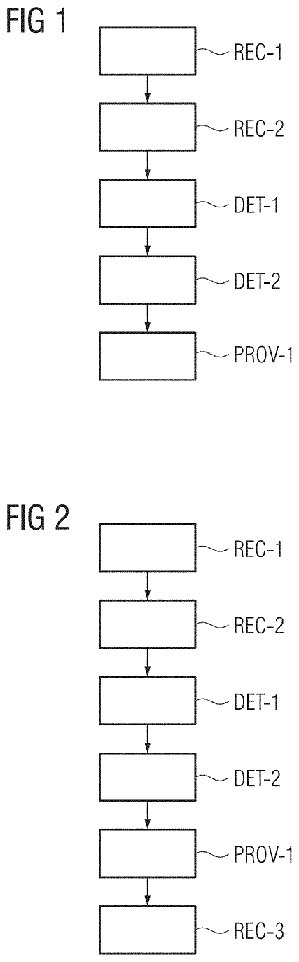

FIG. 1 shows a first example embodiment of a method for allocation of an appointment.

In a method step of receiving REC-1 appointment information Ainf, the appointment information Ainf is received via an interface AU.IN. The appointment information Ainf relates to the appointment. The appointment is a medical appointment. During the medical appointment, in particular a medical examination and/or a medical intervention and/or medical imaging and/or a medical diagnosis is carried out. The medical appointment can be, in particular, a vaccination appointment. In other words, the appointment can relate to dispensing of a vaccine. The vaccine can be administered against an infectious disease. The vaccination appointment can be an appointment for administering a first dose of the vaccine (first vaccination) or a second dose of the vaccine (second vaccination or booster vaccine). The vaccine can be designed to prevent an infection with the infectious disease and/or to lessen a progression of the infection with the infectious disease. The infectious disease can be, in particular, Covid-19. The appointment can be, in particular, in a hospital and/or with a family doctor and/or with a radiologist and/or at a vaccination center and/or in a care facility, etc. The appointment is carried out by medical staff, for example a doctor, a radiologist, a medical assistant etc.

In alternative embodiments, the appointment can relate to any other situations. For example, the appointment can relate to an appointment in a citizen center. Alternatively, the appointment can relate to an appointment with an attorney. Alternatively, the appointment can relate to a shopping appointment.

The appointment information Ainf includes information about a location of the appointment. The location of the appointment indicates where the appointment takes place. As described above, the location can be, for example, a hospital, a family practice, a radiology department, a vaccination center and/or a care facility. For example, the appointment information Ainf can include the address of the location at which the appointment takes place. Alternatively or in addition, the appointment information Ainf can include GPS coordinates of the location at which the appointment takes place. In particular, the appointment information Ainf can include the GPS coordinates in the form of a GPX file. In other words, the information about the location of the appointment can be received in the form of the address and/or in the form of GPS coordinates with the appointment information Ainf.
In a method step of receiving REC-2 at least one first item of patient information Pinf, the at least one first item of patient information Pinf of a first patient is received with the interface AU.IF. In particular, a first item of patient information can be received for a plurality of first patients. A first item of patient information can be assigned to any first patient of the plurality of first patients.

The first patient can be recorded or registered for the appointment in particular. In other words, the first patient can be interested in the appointment. If the appointment is not a medical appointment, the first patient can be a client or a customer or a citizen, etc. The first item of patient information Pinf refers to the first patient. In other words, the first item of patient information Pinf includes at least one item of information about the first patient. The first item of patient information Pinf includes information about a position of the first patient. The information about the position of the first patient indicates where the first patient is at the instant of receiving REC-2 the first item of patient information Pinf. The first item of patient information Pinf can comprise, in particular, the address the position of the first patient. Alternatively or in addition, the first item of patient information Pinf can include the position of the first patient as GPS coordinates, in particular in a GPX file. In other words, the information about the position of the first patient can be received in the form of an address and/or in the form of GPS coordinates with the first item of patient information Pinf. The information about the position of the first patient can be provided, in particular, by a mobile device of the first patient. The first patient can have the mobile device with them at all times. The mobile device can be, in particular, a smartphone and/or a tablet and/or a mobile phone or cell phone. The position of the first patient can be determined and provided via the location determination of the mobile device. Alternatively or in addition, the position of the first patient can be determined and provided by the radio cell into which the mobile device of the first patient is dialed at the instant of receiving REC-2 the first item of patient information Pinf.

In embodiments, the first item of patient information Pinf can also include information about the mobility of the first patient. The information about mobility of the first patient can indicate how the first patient can arrive at the location of the appointment. For example, the information about the mobility of the first patient can indicate that the patient will arrive at the location of the appointment by bicycle, car, bus or train, by taxi and/or on foot from their position. Alternatively or in addition, the information about the mobility of the first patient can indicate whether the first patient is limited in terms of their mobility compared to a mean of the population. In other words, the information about the mobility can include information about the individual mobility of the first patient. For example, the individual mobility can take into account the age of the first patient. Alternatively or in addition, the individual mobility can take into account whether the first patient has difficulty walking and/or is reliant on a wheelchair. The information about the mobility of the first patient can be provided by the first patient. Alternatively or in addition, the information about the individual mobility of the first patient can be derived from health information and/or patient information about the first patient. For example, the information about the individual mobility of the patient can be derived from the age and/or a pre-existing condition of the patient.

In a method step of determining DET-1 a first distance, the first distance between the location of the appointment and the position of the first patient is determined. The first distance is determined on the basis of the information about the location of the appointment and the information about the position of the patient. The first distance can be determined as a linear distance between the location of the appointment and the position of the first patient. Alternatively, when determining DET-1 the first distance, a map can be used as the basis. In particular, the first distance can then be determined as a function of routes and streets, which the first patient can use to arrive from their position at the location of the appointment. In particular, the first distance can then be determined with a geoinformation system on the basis of the GPS coordinates of the location of the appointment and the position of the first patient. For example, the first distance can be determined via Google Maps and/or Open Street Maps.

If the first item of patient information Pinf includes information about the mobility of the first patient, the first distance can be determined as a function of this information. For example, it is possible to take into account that the first patient, if they arrive at the appointment by car, can drive on a motorway whereas this is not possible with a bicycle. The first distance can thus depend on the mobility of the first patient.

In a method step of determining DET-2 a first appointment invitation Ainv, the first appointment invitation Ainv is determined as a function of the first distance. In particular, the first appointment invitation Ainv can only be determined if the first distance undershoots a threshold value. In this way it is possible to ensure that the first patient can turn up promptly to the appointment. In particular, it is possible to prevent, for example a patient who is on vacation and currently cannot turn up to the appointment from being invited.

The first appointment invitation Ainv is designed to inform the first patient about the appointment. In particular, the first appointment invitation can include the information about the location of the appointment. Alternatively or in addition, the first appointment invitation can include information about a time at which the appointment takes place. The information about the time at which the appointment takes place indicates when the appointment begins. In addition, the information about the time at which the appointment takes place can indicate how long the appointment lasts or when the appointment ends. In other words, the first patient can be informed via the first appointment invitation about when and where the appointment available for selection is to take place.

In a method step of providing PROV-1 the first appointment invitation Ainv, the first appointment invitation Ainv is provided via the interface. In particular, the first patient is provided with the first appointment invitation Ainv. For example, the first patient can be provided with the first appointment invitation Ainv via a pop-up window and/or via an SMS and/or via an e-mail and/or via a telephone call. In particular, the first patient can be provided with the first appointment invitation AInv on their mobile device. In particular, the first appointment invitation AInv can be displayed to the first patient. In other words, providing PROV-1 the first appointment invitation AInf can include displaying the first appointment invitation AInf.

In embodiments of the present invention, the first appointment invitation AInv can be provided for the duration of a predefined period. The predefined period can be chosen in such a way that it is ensured that the first patient has enough time after the expiry of the predefined period to arrive promptly at the location of the appointment. In addition, the predefined period can be chosen in such a way that the medical staff has enough time after the expiry of the predefined period to prepare for the appointment. After expiry of the predefined period it is no longer possible to accept the first appointment invitation. The first patient can be informed, in particular in the pop-up window and/or by SMS and/or by e-mail, about the expiry of the predefined period. In particular, the remaining time of the predefined period can be rundown in the pop-up window.

If the first item of patient information PInf includes information about the mobility of the first patient, in embodiments of the present invention, the method steps of determining DET-1 the first appointment invitation AInv and of providing PROV-1 the first appointment invitation AInv can only be carried out when the first distance undershoots a first threshold value. The first threshold value depends on the mobility of the first patient. The first threshold value is chosen or determined or defined in such a way that the first patient can cover the distance given by the first threshold value within a defined journey time interval. In other words, a journey time interval can be given, which defines how long the first patient may take to arrive at the location of the appointment. The journey time interval can depend on a current instant and a time at which the appointment is to take place. The first threshold value can be derived from the journey time interval and the mobility of the first patient. The first threshold value can depend in embodiments on a bus or train timetable. The first threshold value can thus be greater if the first patient can cover a great distance quickly than if they can cover a shorter distance in the same time.

FIG. 2 shows a second example embodiment of a method for allocation of an appointment.

The method steps of receiving REC-1 appointment information AInf, of receiving REC-2 at least a first item of patient information PInf, of determining DET-1 a first distance, of determining DET-2 a first appointment invitation AInv and of providing PROV-1 the first appointment invitation AInv are formed analogously to the description relating to FIG. 1.

The method also includes a method step of receiving REC-3 confirmation information. In the method step of receiving REC-3 the confirmation information, the confirmation information is received if the first patient accepts the first appointment invitation AInv. The confirmation information indicates that the appointment or the first appointment invitation has been accepted. It is possible to derive from the confirmation information that the appointment is no longer available. The confirmation information can include, in particular, information about the first patient who has accepted the first appointment invitation AInv. For example, the confirmation information can include the age of the first patient and/or a pre-existing condition of the first patient.

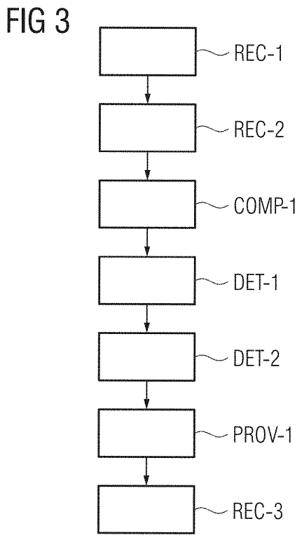

FIG. 3 shows a third example embodiment of a method for allocation of an appointment.

The method steps of receiving REC-1 appointment information AInf, of receiving REC-2 at least a first item of patient information PInf, of determining DET-1 a first distance, of determining DET-2 a first appointment invitation AInv and of providing PROV-1 the first appointment invitation AInv are formed analogously to the description relating to FIG. 1. The method step of receiving REC-3 confirmation information can be formed analogously to the description relating to FIG. 2.

In this example embodiment, the appointment information AInf includes information about a type of appointment. In this example embodiment, the first item of patient information PInf includes information about a type of appointment sought. The type of appointment or appointment sought describes to what the appointment or the appointment sought refers. In other words, the type of appointment or the type of appointment sought describes what is to be carried out or to occur during the appointment or during the appointment sought refers. For example, the information about the type of appointment or the appointment sought can include a description of the appointment or of the appointment sought. The description can read, for example “Vaccination appointment with vaccine” or “MRI scan knee” or “Cardiac sonography” or “Anamnesis”, etc. The description can be selected from a predefined list. Alternatively, the description can include a free text description. The information about the type of appointment can in particular be added by the medical staff to the appointment information AInf by selecting from the list or inputting the text. The information about the type of appointment sought can in particular be added by the first patient to the first item of patient information PInf by selecting from the list or inputting the text. Alternatively, the information about the type of appointment or about the type of appointment sought can include a code, which describes the appointment. The code can be, for example, an ICD code or be based on this. The code can be selected or input accordingly by the medical staff or by the first patient.

The method includes a method step of comparing COMP-1 the type of appointment and the type of appointment sought. The method steps of determining DET-1 the first distance, of determining DET-2 the first appointment invitation AInv and of providing PROV-1 the first appointment invitation AInv are carried out if the comparison produces a match. In other words, said method steps are only carried out when the first patient is also seeking an appointment, which matches the appointment being offered according to the appointment information AInf. If the type of appointment or appointment sought is indicated by a selection from a list or via a predefined code, comparing COMP-1 can include simple comparing of the information about the type of appointment and the information about the type of appointment sought. If the type of appointment and/or the type of appointment sought is indicated by a free text, comparing COMP-1 can include applying a trained function, in particular comprising Natural Language Processing.

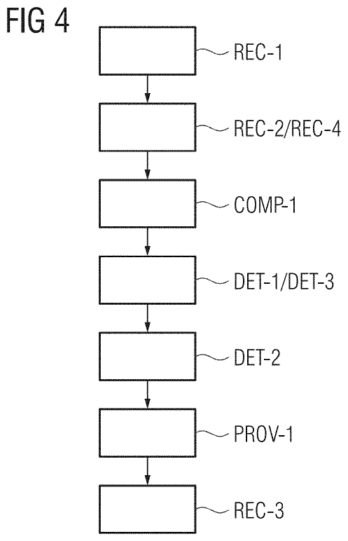

FIG. 4 shows a fourth example embodiment of a method for allocation of an appointment.

The method steps of receiving REC-1 appointment information AInf, of receiving REC-2 at least one first item of patient information PInf, of determining DET-1 a first distance, of determining DET-2 a first appointment invitation AInv and of providing PROV-1 the first appointment invitation AInv are formed analogously to the description relating to FIG. 1. The method step of receiving REC-3 confirmation information can be formed analogously to the description relating to FIG. 2. The method step of comparing COMP-1 the type of appointment and the type of appointment sought is formed analogously to the description relating to FIG. 3.

In the example embodiment, the method includes a method step of receiving REC-4 at least one second item of patient information of a second patient. The method step of receiving REC-4 the at least one second item of patient information is formed analogously to the method step of receiving REC-2 at least one first item of patient information PInf. The second item of patient information in respect of the second patient is formed analogously to the first item of patient information PInf in respect of the first patient. The second item of patient information includes at least one item of information about a position of the second patient. The information about the position of the second patient is formed analogously to the information about the position of the first patient. In particular, the position of the second patient describes where the second patient is at the instant of receiving REC-4 the second item of patient information.

In the example embodiment, the method also includes a method step of determining DET-3 a second distance between the location of the appointment and the position of the second patient. The second distance can be determined analogously to the first distance.

The second item of patient information can include information about the mobility of the second patient. As described above for the first distance, the second distance can depend on the mobility of the second patient. In other words, the mobility of the second patient can be taken into account in the method step of determining DET-3 the second distance.

All further method steps and embodiments, which were described in FIGS. 1 to 3 in respect of the first patient, can be transferred to the second patient.

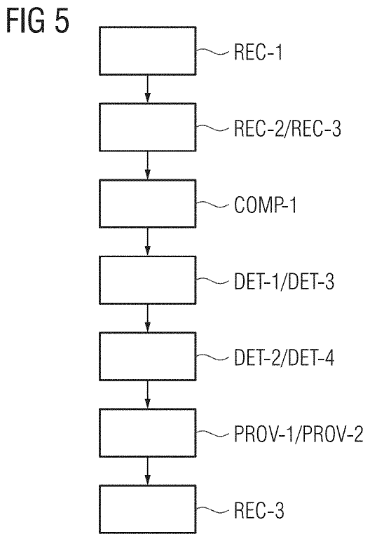

FIG. 5 shows a fifth example embodiment of a method for allocation of an appointment.

The method steps of receiving REC-1 appointment information AInf, of receiving REC-2 at least one first item of patient information PInf, of determining DET-1 a first distance, of determining DET-2 a first appointment invitation AInv and of providing PROV-1 the first appointment invitation AInv are formed analogously to the description relating to FIG. 1. The method step of receiving REC-3 confirmation information can be formed analogously to the description relating to FIG. 2. The method step of comparing COMP-1 the type of appointment and the type of appointment sought is formed analogously to the description relating to FIG. 3. The method steps of receiving REC-4 a second item of patient information and of determining DET-3 a second distance are formed analogously to the description relating to FIG. 4.

In the example embodiment, the method also includes a method step of determining DET-4 a second appointment invitation as a function of the second distance. Analogously to the first appointment invitation AInv, the second appointment invitation can be determined when the second distance undershoots a journey threshold value. The journey threshold value for the first distance can be different from the journey threshold value for the second distance. Alternatively, the journey threshold value can be identical for the first and the second distances. The second appointment invitation is designed to inform the second patient about the appointment. The second appointment invitation can include the appointment information AInf. In particular, the second appointment invitation can include the information about the location of the appointment. Alternatively or in addition, the second appointment invitation can include the information about the time at which the appointment takes place.

In the example embodiment, the method also includes a method step of providing PROV-2 the second appointment invitation. The second patient is provided with the appointment invitation. As described above in respect of the method step of providing PROV-1 the first appointment invitation, the second patient can be provided with the second appointment invitation via a pop-up window and/or via an SMS and/or via an e-mail and/or via a telephone call. In particular, the second patient can be provided with or shown the second appointment invitation on a mobile device of the second patient. In other words, providing PROV-2 the second appointment invitation can include displaying the second appointment invitation.

In embodiments of the present invention, the method steps of determining DET-4 the second appointment invitation and of providing PROV-2 the second appointment invitation are only carried out when the second distance undershoots a second threshold value. The second item of patient information, as described in the description relating to FIG. 4, optionally includes information about the mobility of the second patient. The second threshold value depends on the mobility of the second patient. In particular, the second threshold value depends on the mobility of the second patient in the way that the first threshold value depends on the mobility of the first patient. The first and the second threshold values are thus patient-specific. The first and the second threshold values can thus be different. For example, the second threshold value can be less than the first threshold value if the second patient can arrive at the location of the appointment on foot and the first patient can use a car to arrive at the location of the appointment.

In embodiments of the present invention, the second patient can be provided with the second appointment invitation for the duration of the predefined period. In other words, the second patient can accept or confirm the appointment invitation only within the predefined period.

In the method step of receiving REC-3 the confirmation information, the confirmation information is also received if the second patient accepts the second appointment invitation. In other words, the confirmation information is received if the first and/or the second patient accepts the first or second appointment invitation. The confirmation information can include, in particular, information about which patient has accepted the corresponding appointment invitation. In other words, the confirmation information can include information about which appointment invitation has been accepted. In particular, the confirmation information can include information about the patient who has accepted the appointment invitation. The information about the patient can include, for example, the age and/or a pre-existing condition of the patient.

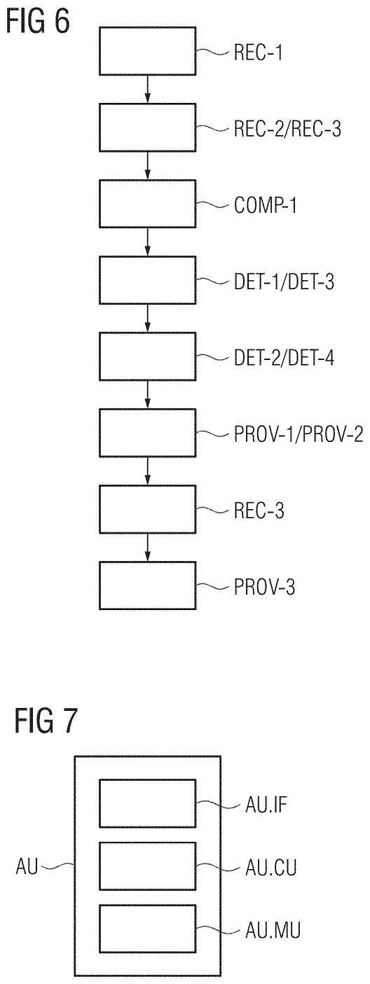

FIG. 6 shows a sixth example embodiment of a method for allocation of an appointment.

The method steps of receiving REC-1 appointment information AInf, of receiving REC-2 at least one first item of patient information PInf, of determining DET-1 a first distance, of determining DET-2 a first appointment invitation AInv and of providing PROV-1 the first appointment invitation AInv are formed analogously to the description relating to FIG. 1. The method step of receiving REC-3 confirmation information can be formed analogously to the description relating to FIG. 2. The method step of comparing COMP-1 the type of appointment and the type of appointment sought is formed analogously to the description relating to FIG. 3. The method steps of receiving REC-4 a second item of patient information and of determining DET-3 a second distance are formed analogously to the description relating to FIG. 4. The method steps of determining DET-4 a second appointment invitation and of providing PROV-2 the second appointment invitation are formed analogously to the description relating to FIG. 5.

In a method step of providing PROV-3 allocation information, the allocation information is provided as soon as the confirmation information has been received. The allocation information can inform a patient if another patient has already successfully accepted the corresponding invitation for the appointment. In particular, the patient can be informed via the allocation information that the appointment is no longer available or that it has already been allocated. For example, the first patient can be provided with the allocation information if the second patient accepts the second appointment invitation and the appointment is thus allocated. Providing PROV-3 the allocation information can include, for example, withdrawal of the first and/or second appointment invitation AInv. The first and/or second appointment invitation AInv can be withdrawn, for example, by a corresponding pop-up window being closed. Alternatively or in addition, the first and/or second appointment invitation AInv can be withdrawn in that it is no longer possible to accept the first and/or second appointment invitation AInv. Alternatively, the first and/or second patient can be provided with the allocation information by SMS and/or e-mail.

In embodiments of the present invention, the allocation information can be patient-specific. In particular, the allocation information can inform that patient, who successfully accepted their appointment invitation, that they should or can use the appointment. All other patients are informed via the allocation information that the appointment has been allocated and it is no longer possible to accept the corresponding appointment invitation.

If the first and the second patient accept the first or second appointment invitation, a patient can be chosen or determined who should actually use the appointment. This patient can be informed via the allocation information that they should use the appointment. The other patient can be informed via the allocation information that the appointment has already been allocated. In particular, the patient who accepts their appointment invitation first, can be determined to use the appointment.

Alternatively, the patient who should use the appointment can be determined on the basis of a prioritization of the patient. In particular, the patient who should use the appointment can be determined on the basis of the prioritization of the patient if the first and the second patient accept the first or second appointment invitation simultaneously. For this, the first item of patient information PInf includes a first item of prioritization information of the first patient in respect of the appointment. For this, the second item of patient information includes a second item of prioritization information of the second patient in respect of the appointment.

More than one patient group can be determined for the prioritization. The first and the second patients can each be assigned to a patient group. A patient group is defined by at least one particular property, which a patient must exhibit so they can be assigned to the corresponding patient group. The patient groups are arranged in an order in respect of their prioritization in respect of the appointment. This means patients who are assigned to a first patient group are prioritized compared to patients who are assigned to a second patient group when providing the appointment invitation and/or when determining who should use the appointment. The patient groups can divide the patients for example in respect of their waiting time for the appointment and/or in respect of their age and/or in respect of their pre-existing conditions and/or in respect of their occupation and/or in respect of their social environment. In other words, the at least one property can be, for example, a specification of the waiting time for the appointment and/or of the age and/or of the occupation and/or of the pre-existing conditions and/or of the social environment. For example, the first patient can have been waiting longer for the appointment than the second patient. The first patient can then be given preference to use the appointment if both patients accept their corresponding appointment invitations. Alternatively or in addition, the first patient can be older than the second patient. The first patient can then be given preference to use the appointment if both patients accept their corresponding appointment invitations.

The prioritization or the division into patient groups can depend, in particular, on the type of appointment. If the appointment relates, for example, to allocation of a vaccine, or if the appointment is a vaccination appointment, the prioritization can depend on an occurrence of infection in an environment of the first and/or second patient. The occurrence of infection describes a spread of the infectious disease, which can be reduced with the vaccine. The occurrence of infection can be mapped, for example, by a number of new infections and/or by an incidence and/or by intensive care bed occupation and/or by a reproduction rate. The environment of the first or second patient can include, in particular, the region in which the first or second patient preferably resides. For example, the environment can include at least a domicile and a place of work of the first or second patient. Alternatively or in addition, the environment of the first or second patient can include a region with a radius of 10, 15, 20, 30, 50 km around the domicile of the first or second patient. Alternatively or in addition, the environment of the first or second patient can include a district or an urban area, which includes the domicile of the first or second patient. If the occurrence of infection is higher and/or more uncontrollable in the environment of the first patient than in the environment of the second patient, the first patient can then be given preference to use the appointment if both patients accept their corresponding appointment invitations.

The first and/or second item of prioritization information can include information about to which patient group the first or second patient is assigned. Alternatively or in addition, the first and/or second item of prioritization information can comprise the property, which allows an assignment of the first and/or second patient to a patient group. For example, the first and/or second item of prioritization information can include the age and/or the occupation and/or the pre-existing conditions and/or the social environment of the first or second patient. In other words, the first and/or second item of prioritization information can include a prioritization of the first or second patient in respect of their age, their occupation, their pre-existing conditions and/or their social environment. The prioritization can be based on the assignment to patient groups. The wording “the first and/or second item of prioritization information includes the age or the occupation or the pre-existing conditions or the social environment of the first or second patient” used below also includes the wording “the first and/or second item of prioritization information includes a prioritization in respect of the age or the occupation or the pre-existing conditions or the social environment of the first or second patient”.

The first and/or second item of prioritization information can include the age of the first or second patient, for example in the form of the date of birth. Alternatively or in addition, the first and/or second item of prioritization information can include the age in the form of a natural number or a numerical value, which represents the age of the first or second patient.

The first and/or second item of prioritization information can include the occupation of the first or second patient according to a division into general occupational fields. The occupational fields can read, for example, “Care”, “Teaching”, “Medicine”, “Support”, “Office”, “Manufacturing sector”, etc. Alternatively or in addition, the first and/or second item of prioritization information can include a free text designation for the occupation of the first or second patient. The assignment of the first and/or second patient to the patient groups can then include by applying a trained function to the first or second item of prioritization information. The trained function can be based, in particular, on Natural Language Processing.

The first and/or second item of prioritization information can include the pre-existing conditions of the first or second patient, in particular in the form of an ICD code.

The first and/or second item of prioritization information can include information about the social environment of the first or second patient, for example in the form of a description of a housing situation of the first or second patient. The description the housing situation can include, for example, a number of members of a household of the first and/or second patient. Alternatively or in addition, the description of the housing situation can include a grouping of the housing situation of the first or second patient, for example into “Single-family house”, “Apartment”, “Communal accommodation”.

In embodiments of the present invention, the method steps of determining DET-2 the first appointment invitation AInv, of providing PROV-1 the first appointment invitation AInv, of determining DET-4 the second appointment invitation and of providing PROV-2 the second appointment invitation can take place as a function of the first and/or second item of prioritization information. In particular, an appointment invitation can be determined and provided only for patients who can be assigned on the basis of the corresponding prioritization information to at least one particular patient group.

For example, a vaccination appointment can be allocated only to patients over 80. The first item of prioritization information includes, for example, information to the effect that the first patient is 90 years old. The second item of prioritization information includes, for example, information to the effect that the second patient is 50 years old. The method steps of determining DET-4 the second appointment invitation and of providing PROV-2 the second appointment invitation are then not carried out since, on the basis of the prioritization information, the second patient is not entitled to use a vaccination appointment. The method steps of determining DET-2 the first appointment invitation AInv and of providing PROV-1 the first appointment invitation AInv are carried out as described.

FIG. 7 shows an appointment allocation unit AU for allocation of an appointment.

The illustrated appointment allocation unit AU for allocation of an appointment is designed to carry out an inventive method for allocation of an appointment, in particular according to the description relating to FIGS. 1 to 6. The appointment allocation unit AU includes an interface AU.IF, a computing unit AU.CU and a memory unit AU.MU.

The appointment allocation unit AU can be, in particular, a computer, a microcontroller or an integrated circuit (IC). Alternatively, the appointment allocation unit AU can be a real or virtual computer network (a technical name for a real computer network is “cluster”, a technical name for a virtual computer network is “cloud”). The appointment allocation unit AU can be designed as a virtual system, which is run on a computer or a real computer network or a virtual computer network (a technical name is “virtualization”).

The interface AU.IF can be a hardware or software interface (for example a PCI bus, USB or Firewire). The computing unit AU.CU can include hardware and/or software constituent parts, for example a microprocessor or what is known as an FPGA (Field Programmable Gate Array). The memory unit AU.MU can be designed as a Random Access Memory (RAM) or as a permanent mass storage device (hard disk, USB stick, SD card, Solid State Disk (SSD).

The interface AU.IF can include, in particular, a plurality of sub-interfaces, which carry out different method steps of the respect inventive method. In other words, the interface AU.IF can be designed as a plurality of interfaces AU.IF. The computing unit AU.CU can include, in particular, a plurality of sub-computing units, which carry out different method steps of the respective inventive method. In other words, the computing unit AU.CU can be designed as a plurality of computing units AU.CU.

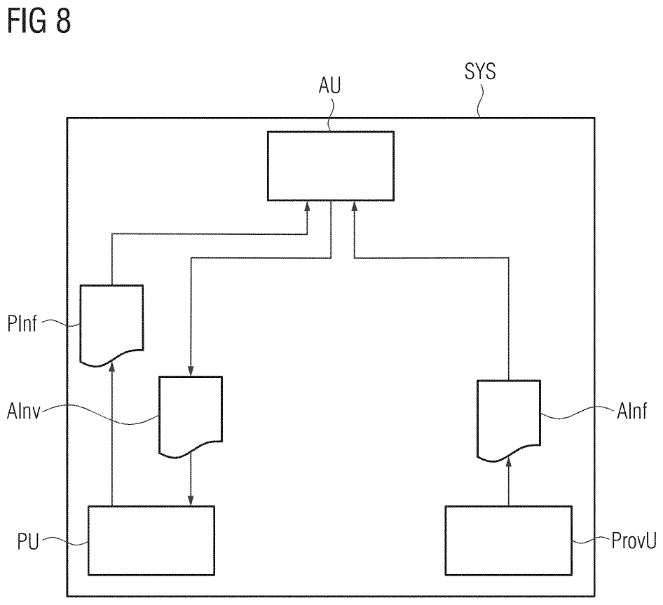

FIG. 8 shows an appointment allocation system SYS for allocation of an appointment.

The appointment allocation system SYS includes an appointment allocation unit AU according to the description relating to FIG. 7. The appointment allocation system SYS also includes at least one first patient terminal PU and a provision unit ProvU.

The first patient terminal PU is designed for sending the first item of patient information PInf of a first patient and for receiving the first appointment invitation AInv. The first item of patient information PInf and the first appointment invitation AInv are formed according to the descriptions relating to FIGS. 1 to 6.

The provision unit ProvU is designed for sending the appointment information AInf. The appointment information AInf is formed according to the descriptions relating to FIGS. 1 to 6.

The first patient terminal PU is the mobile device of the first patient. The first patient terminal PU is, in particular, a smartphone and/or a tablet and/or a mobile phone or cell phone. Alternatively, the first patient terminal AU can be a desktop computer and/or a laptop. The first patient terminal PU can display the first appointment invitation AInv via a display unit. The display unit can be a screen or monitor. The first patient terminal PU is designed for receiving a user input by the first patient. The user input can be received, in particular, via an input unit. The input unit can be, for example, a computer keyboard and/or a computer mouse and/or a touchpad and/or a touchscreen. In particular, the first patient can provide the first item of patient information PInf via the user input.

The provision unit ProvU can be, in particular, a smartphone and/or a tablet and/or a desktop computer and/or a laptop belonging to the medical staff. The provision unit ProvU can be designed to receive a user input of the medical staff. The user input can be received via a receiving unit. The receiving unit can be designed, in particular, as a computer keyboard and/or a computer mouse and/or a touchpad and/or a touchscreen. In embodiments of the present invention, the receiving unit can include a speech recognition system or a dictation system. In particular, the medical staff can provide the provision unit ProvU with the appointment information AInf via a user input. The provision unit ProvU can also include a display unit in order to display the appointment information AInf to the medical staff. The display unit can be, in particular, a screen and/or a monitor. The provision unit ProvU can have, in particular, access to a server. A database is stored on the server. The database includes the appointment information AInf and at least the first item of patient information PInf.

In embodiments of the present invention, the appointment allocation system SYS can also include at least one second patient terminal, which is designed for sending the second item of patient information and for receiving the second item of appointment information. The second patient terminal can be designed analogously to the first patient terminal.

For the sake of clarity, it is to be understood that the use of “a” or “an” throughout this application does not exclude a plurality, and “comprising” does not exclude other steps or elements. The mention of a “unit” or a “module” does not preclude the use of more than one unit or module.

The drawings are to be regarded as being schematic representations and elements illustrated in the drawings are not necessarily shown to scale. Rather, the various elements are represented such that their function and general purpose become apparent to a person skilled in the art. Any connection or coupling between functional blocks, devices, components, or other physical or functional units shown in the drawings or described herein may also be implemented by an indirect connection or coupling. A coupling between components may also be established over a wireless connection. Functional blocks may be implemented in hardware, firmware, software, or a combination thereof.

It will be understood that, although the terms first, second, etc. may be used herein to describe various elements, components, regions, layers, and/or sections, these elements, components, regions, layers, and/or sections, should not be limited by these terms. These terms are only used to distinguish one element from another. For example, a first element could be termed a second element, and, similarly, a second element could be termed a first element, without departing from the scope of example embodiments. As used herein, the term “and/or,” includes any and all combinations of one or more of the associated listed items. The phrase “at least one of” has the same meaning as “and/or”.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below,” “beneath,” or “under,” other elements or features would then be oriented “above” the other elements or features. Thus, the example terms “below” and “under” may encompass both an orientation of above and below. The device may be otherwise oriented (rotated 90 degrees or at other orientations) and the spatially relative descriptors used herein interpreted accordingly. In addition, when an element is referred to as being “between” two elements, the element may be the only element between the two elements, or one or more other intervening elements may be present.

Spatial and functional relationships between elements (for example, between modules) are described using various terms, including “connected,” “engaged,” “interfaced,” and “coupled.” Unless explicitly described as being “direct,” when a relationship between first and second elements is described in the disclosure, that relationship encompasses a direct relationship where no other intervening elements are present between the first and second elements, and also an indirect relationship where one or more intervening elements are present (either spatially or functionally) between the first and second elements. In contrast, when an element is referred to as being “directly” connected, engaged, interfaced, or coupled to another element, there are no intervening elements present. Other words used to describe the relationship between elements should be interpreted in a like fashion (e.g., “between,” versus “directly between,” “adjacent,” versus “directly adjacent,” etc.).

The terminology used herein is for the purpose of describing particular embodiments only and is not intended to be limiting of example embodiments. As used herein, the singular forms “a,” “an,” and “the,” are intended to include the plural forms as well, unless the context clearly indicates otherwise. As used herein, the terms “and/or” and “at least one of” include any and all combinations of one or more of the associated listed items. It will be further understood that the terms “comprises,” “comprising,” “includes,” and/or “including,” when used herein, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items. Expressions such as “at least one of,” when preceding a list of elements, modify the entire list of elements and do not modify the individual elements of the list. Also, the term “example” is intended to refer to an example or illustration.

When an element is referred to as being “on,” “connected to,” “coupled to,” or “adjacent to,” another element, the element may be directly on, connected to, coupled to, or adjacent to, the other element, or one or more other intervening elements may be present. In contrast, when an element is referred to as being “directly on,” “directly connected to,” “directly coupled to,” or “immediately adjacent to,” another element there are no intervening elements present.

It should also be noted that in some alternative implementations, the functions/acts noted may occur out of the order noted in the figures. For example, two figures shown in succession may in fact be executed substantially concurrently or may sometimes be executed in the reverse order, depending upon the functionality/acts involved.

Unless otherwise defined, all terms (including technical and scientific terms) used herein have the same meaning as commonly understood by one of ordinary skill in the art to which example embodiments belong. It will be further understood that terms, e.g., those defined in commonly used dictionaries, should be interpreted as having a meaning that is consistent with their meaning in the context of the relevant art and will not be interpreted in an idealized or overly formal sense unless expressly so defined herein.

It is noted that some example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed above. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order. Although the flowcharts describe the operations as sequential processes, many of the operations may be performed in parallel, concurrently or simultaneously. In addition, the order of operations may be re-arranged. The processes may be terminated when their operations are completed, but may also have additional steps not included in the figure. The processes may correspond to methods, functions, procedures, subroutines, subprograms, etc.

Specific structural and functional details disclosed herein are merely representative for purposes of describing example embodiments. The present invention may, however, be embodied in many alternate forms and should not be construed as limited to only the embodiments set forth herein.

Units and/or devices according to one or more example embodiments may be implemented using hardware, software, and/or a combination thereof. For example, hardware devices may be implemented using processing circuitry such as, but not limited to, a processor, Central Processing Unit (CPU), a controller, an arithmetic logic unit (ALU), a digital signal processor, a microcomputer, a field programmable gate array (FPGA), a System-on-Chip (SoC), a programmable logic unit, a microprocessor, or any other device capable of responding to and executing instructions in a defined manner. Portions of the example embodiments and corresponding detailed description may be presented in terms of software, or algorithms and symbolic representations of operation on data bits within a computer memory. These descriptions and representations are the ones by which those of ordinary skill in the art effectively convey the substance of their work to others of ordinary skill in the art. An algorithm, as the term is used here, and as it is used generally, is conceived to be a self-consistent sequence of steps leading to a desired result. The steps are those requiring physical manipulations of physical quantities. Usually, though not necessarily, these quantities take the form of optical, electrical, or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be borne in mind that all of these and similar terms are to be associated with the appropriate physical quantities and are merely convenient labels applied to these quantities. Unless specifically stated otherwise, or as is apparent from the discussion, terms such as “processing” or “computing” or “calculating” or “determining” of “displaying” or the like, refer to the action and processes of a computer system, or similar electronic computing device/hardware, that manipulates and transforms data represented as physical, electronic quantities within the computer system's registers and memories into other data similarly represented as physical quantities within the computer system memories or registers or other such information storage, transmission or display devices.

In this application, including the definitions below, the term ‘module’ or the term ‘controller’ may be replaced with the term ‘circuit.’ The term ‘module’ may refer to, be part of, or include processor hardware (shared, dedicated, or group) that executes code and memory hardware (shared, dedicated, or group) that stores code executed by the processor hardware.

The module may include one or more interface circuits. In some examples, the interface circuits may include wired or wireless interfaces that are connected to a local area network (LAN), the Internet, a wide area network (WAN), or combinations thereof. The functionality of any given module of the present disclosure may be distributed among multiple modules that are connected via interface circuits. For example, multiple modules may allow load balancing. In a further example, a server (also known as remote, or cloud) module may accomplish some functionality on behalf of a client module.

Software may include a computer program, program code, instructions, or some combination thereof, for independently or collectively instructing or configuring a hardware device to operate as desired. The computer program and/or program code may include program or computer-readable instructions, software components, software modules, data files, data structures, and/or the like, capable of being implemented by one or more hardware devices, such as one or more of the hardware devices mentioned above. Examples of program code include both machine code produced by a compiler and higher level program code that is executed using an interpreter.

For example, when a hardware device is a computer processing device (e.g., a processor, Central Processing Unit (CPU), a controller, an arithmetic logic unit (ALU), a digital signal processor, a microcomputer, a microprocessor, etc.), the computer processing device may be configured to carry out program code by performing arithmetical, logical, and input/output operations, according to the program code. Once the program code is loaded into a computer processing device, the computer processing device may be programmed to perform the program code, thereby transforming the computer processing device into a special purpose computer processing device. In a more specific example, when the program code is loaded into a processor, the processor becomes programmed to perform the program code and operations corresponding thereto, thereby transforming the processor into a special purpose processor.

Software and/or data may be embodied permanently or temporarily in any type of machine, component, physical or virtual equipment, or computer storage medium or device, capable of providing instructions or data to, or being interpreted by, a hardware device. The software also may be distributed over network coupled computer systems so that the software is stored and executed in a distributed fashion. In particular, for example, software and data may be stored by one or more computer readable recording mediums, including the tangible or non-transitory computer-readable storage media discussed herein.

Even further, any of the disclosed methods may be embodied in the form of a program or software. The program or software may be stored on a non-transitory computer readable medium and is adapted to perform any one of the aforementioned methods when run on a computer device (a device including a processor). Thus, the non-transitory, tangible computer readable medium, is adapted to store information and is adapted to interact with a data processing facility or computer device to execute the program of any of the above mentioned embodiments and/or to perform the method of any of the above mentioned embodiments.

Example embodiments may be described with reference to acts and symbolic representations of operations (e.g., in the form of flow charts, flow diagrams, data flow diagrams, structure diagrams, block diagrams, etc.) that may be implemented in conjunction with units and/or devices discussed in more detail below. Although discussed in a particularly manner, a function or operation specified in a specific block may be performed differently from the flow specified in a flowchart, flow diagram, etc. For example, functions or operations illustrated as being performed serially in two consecutive blocks may actually be performed simultaneously, or in some cases be performed in reverse order.

According to one or more example embodiments, computer processing devices may be described as including various functional units that perform various operations and/or functions to increase the clarity of the description. However, computer processing devices are not intended to be limited to these functional units. For example, in one or more example embodiments, the various operations and/or functions of the functional units may be performed by other ones of the functional units. Further, the computer processing devices may perform the operations and/or functions of the various functional units without sub-dividing the operations and/or functions of the computer processing units into these various functional units.

Units and/or devices according to one or more example embodiments may also include one or more storage devices. The one or more storage devices may be tangible or non-transitory computer-readable storage media, such as random access memory (RAM), read only memory (ROM), a permanent mass storage device (such as a disk drive), solid state (e.g., NAND flash) device, and/or any other like data storage mechanism capable of storing and recording data. The one or more storage devices may be configured to store computer programs, program code, instructions, or some combination thereof, for one or more operating systems and/or for implementing the example embodiments described herein. The computer programs, program code, instructions, or some combination thereof, may also be loaded from a separate computer readable storage medium into the one or more storage devices and/or one or more computer processing devices using a drive mechanism. Such separate computer readable storage medium may include a Universal Serial Bus (USB) flash drive, a memory stick, a Blu-ray/DVD/CD-ROM drive, a memory card, and/or other like computer readable storage media. The computer programs, program code, instructions, or some combination thereof, may be loaded into the one or more storage devices and/or the one or more computer processing devices from a remote data storage device via a network interface, rather than via a local computer readable storage medium. Additionally, the computer programs, program code, instructions, or some combination thereof, may be loaded into the one or more storage devices and/or the one or more processors from a remote computing system that is configured to transfer and/or distribute the computer programs, program code, instructions, or some combination thereof, over a network. The remote computing system may transfer and/or distribute the computer programs, program code, instructions, or some combination thereof, via a wired interface, an air interface, and/or any other like medium.

The one or more hardware devices, the one or more storage devices, and/or the computer programs, program code, instructions, or some combination thereof, may be specially designed and constructed for the purposes of the example embodiments, or they may be known devices that are altered and/or modified for the purposes of example embodiments.

A hardware device, such as a computer processing device, may run an operating system (OS) and one or more software applications that run on the OS. The computer processing device also may access, store, manipulate, process, and create data in response to execution of the software. For simplicity, one or more example embodiments may be exemplified as a computer processing device or processor; however, one skilled in the art will appreciate that a hardware device may include multiple processing elements or processors and multiple types of processing elements or processors. For example, a hardware device may include multiple processors or a processor and a controller. In addition, other processing configurations are possible, such as parallel processors.

The computer programs include processor-executable instructions that are stored on at least one non-transitory computer-readable medium (memory). The computer programs may also include or rely on stored data. The computer programs may encompass a basic input/output system (BIOS) that interacts with hardware of the special purpose computer, device drivers that interact with particular devices of the special purpose computer, one or more operating systems, user applications, background services, background applications, etc. As such, the one or more processors may be configured to execute the processor executable instructions.

The computer programs may include: (i) descriptive text to be parsed, such as HTML (hypertext markup language) or XML (extensible markup language), (ii) assembly code, (iii) object code generated from source code by a compiler, (iv) source code for execution by an interpreter, (v) source code for compilation and execution by a just-in-time compiler, etc. As examples only, source code may be written using syntax from languages including C, C++, C#, Objective-C, Haskell, Go, SQL, R, Lisp, Java®, Fortran, Perl, Pascal, Curl, OCaml, Javascript®, HTML5, Ada, ASP (active server pages), PHP, Scala, Eiffel, Smalltalk, Erlang, Ruby, Flash®, Visual Basic®, Lua, and Python®.

Further, at least one example embodiment relates to the non-transitory computer-readable storage medium including electronically readable control information (processor executable instructions) stored thereon, configured in such that when the storage medium is used in a controller of a device, at least one embodiment of the method may be carried out.

The computer readable medium or storage medium may be a built-in medium installed inside a computer device main body or a removable medium arranged so that it can be separated from the computer device main body. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The term code, as used above, may include software, firmware, and/or microcode, and may refer to programs, routines, functions, classes, data structures, and/or objects. Shared processor hardware encompasses a single microprocessor that executes some or all code from multiple modules. Group processor hardware encompasses a microprocessor that, in combination with additional microprocessors, executes some or all code from one or more modules. References to multiple microprocessors encompass multiple microprocessors on discrete dies, multiple microprocessors on a single die, multiple cores of a single microprocessor, multiple threads of a single microprocessor, or a combination of the above.

Shared memory hardware encompasses a single memory device that stores some or all code from multiple modules. Group memory hardware encompasses a memory device that, in combination with other memory devices, stores some or all code from one or more modules.

The term memory hardware is a subset of the term computer-readable medium. The term computer-readable medium, as used herein, does not encompass transitory electrical or electromagnetic signals propagating through a medium (such as on a carrier wave); the term computer-readable medium is therefore considered tangible and non-transitory. Non-limiting examples of the non-transitory computer-readable medium include, but are not limited to, rewriteable non-volatile memory devices (including, for example flash memory devices, erasable programmable read-only memory devices, or a mask read-only memory devices); volatile memory devices (including, for example static random access memory devices or a dynamic random access memory devices); magnetic storage media (including, for example an analog or digital magnetic tape or a hard disk drive); and optical storage media (including, for example a CD, a DVD, or a Blu-ray Disc). Examples of the media with a built-in rewriteable non-volatile memory, include but are not limited to memory cards; and media with a built-in ROM, including but not limited to ROM cassettes; etc. Furthermore, various information regarding stored images, for example, property information, may be stored in any other form, or it may be provided in other ways.

The apparatuses and methods described in this application may be partially or fully implemented by a special purpose computer created by configuring a general purpose computer to execute one or more particular functions embodied in computer programs. The functional blocks and flowchart elements described above serve as software specifications, which can be translated into the computer programs by the routine work of a skilled technician or programmer.

Although described with reference to specific examples and drawings, modifications, additions and substitutions of example embodiments may be variously made according to the description by those of ordinary skill in the art. For example, the described techniques may be performed in an order different with that of the methods described, and/or components such as the described system, architecture, devices, circuit, and the like, may be connected or combined to be different from the above-described methods, or results may be appropriately achieved by other components or equivalents.

Where it has not explicitly occurred, but expedient and within the meaning of the present invention, individual example embodiments, individual partial aspects or features thereof can be combined with each other or interchanged without departing from the scope of the present invention. Advantages of the present invention described with regard to one example embodiment also apply without being explicit mentioned, and where transferable, to other example embodiments.

What is claimed is:

1. A computer-implemented method for allocation of an appointment, the computer-implemented method comprising:
receiving appointment information, the appointment information including information about a location of the appointment,
receiving at least a first item of patient information for a first patient, the first item of patient information including information about a position of the first patient,
determining a first distance between the location of the appointment and the position of the first patient,
determining a first appointment invitation as a function of the first distance, and
providing the first appointment invitation.

2. The method as claimed in claim 1, wherein
the first item of patient information includes information about a mobility of the first patient,
the determining a first appointment invitation and the providing the first appointment invitation take place only when the first distance is less than a first threshold value, and
the first threshold value depends on the mobility of the first patient.

3. The method as claimed in claim 1, further comprising:
receiving confirmation information in response to the first patient accepting the first appointment invitation.

4. The method as claimed in claim 1, wherein the appointment relates to dispensing of a vaccine.

5. The method as claimed in claim 1, wherein
the appointment information includes information about a type of the appointment,
the first item of patient information includes information about a type of appointment sought,
the method further includes comparing the type of the appointment and the type of appointment sought, and
the determining a first distance, the determining a first appointment invitation and the providing the first appointment invitation are carried out in response to the comparing producing a match.

6. The method as claimed in claim 1, wherein the first appointment invitation is provided for the duration of a period.

7. The method as claimed in claim 1, wherein
the appointment information includes information about a time at which the appointment is to take place, and
the first appointment invitation includes at least one of the information about the location of the appointment or the time at which the appointment is to take place.

8. The method as claimed in claim 1, further comprising:
receiving at least one second item of patient information for a second patient, the at least one second item of patient information including information about a position of the second patient, and
determining a second distance between the location of the appointment and the position of the second patient.

9. The method as claimed in claim 8, further comprising:
determining a second appointment invitation as a function of the second distance, and
providing the second appointment invitation.

10. The method as claimed in claim 9, wherein
the determining a second appointment invitation and the providing the second appointment invitation take place only when the second distance is less than a second threshold value, and
the second threshold value depends on a mobility of the second patient.

11. The method as claimed in claim 9, further comprising:
receiving first confirmation information when the first patient accepts the first appointment invitation, and
receiving second confirmation information when the second patient accepts the second appointment invitation.

12. The method as claimed in claim 11, further comprising:
providing allocation information when the first confirmation information or the second confirmation information is received.

13. The method as claimed in claim 9, wherein
at least one of (i) the first item of patient information includes a first item of prioritization information of the first patient in respect of the appointment or (ii) the second item of patient information includes a second item of prioritization information of the second patient in respect of the appointment, and
the determining a first appointment invitation, the providing the first appointment invitation, the determining a second appointment invitation and the providing the second appointment invitation take place as a function of at least one of the first item of prioritization information or the second item of prioritization information.

14. The method as claimed in claim 13, wherein at least one of the first item of prioritization information or the second item of prioritization information includes a prioritization of the first patient or the second patient in respect of at least one of age, occupation, pre-existing conditions or social environment.

15. The method as claimed in claim 13, wherein
the appointment relates to dispensing of a vaccine, and
the at least one of the first item of prioritization information or the second item of prioritization information includes information about an occurrence of infection of an infectious disease reduceable by the vaccine in an environment of the first patient or the second patient.

16. An appointment allocation unit for allocation of an appointment, the appointment allocation unit comprising:
an interface; and
a computing unit,
wherein the interface is configured to
receive appointment information, the appointment information including information about a location of the appointment,
receive at least a first item of patient information of a first patient, the first item of patient information including information about a position of the first patient, and
provide a first appointment invitation as a function of the first item of patient information, the first appointment invitation being provided only in response to a first distance between the location of the appointment and the position of the first patient being less than a first threshold value; and
wherein the computing unit is configured to
determine the first distance between the location of the appointment and the position of the first patient, and
determine the first appointment invitation as a function of at least one of the first item of patient information or the first distance.

17. An appointment allocation system for allocation of an appointment, the appointment allocation system comprising:
the appointment allocation unit as claimed in claim 16;
at least one first patient terminal configured to send the first item of patient information of the first patient, and to receive the first appointment invitation; and
a provision unit configured to send the appointment information.

18. A non-transitory computer program product with a computer program directly loadable into a memory unit of an appointment allocation unit, the computer program including program segments that, when executed by the appointment allocation unit, carry out the method as claimed in claim 1.

19. A non-transitory computer-readable storage medium storing program segments, which are readable and executable by an appointment allocation unit, the program segments, when executed by the appointment allocation unit, cause the appointment allocation unit to perform the method as claimed in claim 1.

20. The method as claimed in claim 9, wherein the second appointment invitation is provided for the duration of a period.
